(19) United States
(12) Patent Application Publication
(10) Pub. No.: US 2004/0215247 A1
(43) Pub. Date: Oct. 28, 2004

(54) Method and device for detecting an anomaly in the cardiac activity of a patient

(76) Inventor: Armin Bolz, Buckenhof (DE)

Correspondence Address: DILWORTH & BARRESE, LLP, 333 EARLE OVINGTON BLVD., UNIONDALE, NY, 11553, US

(21) Appl. No.: 10/755,534

(22) Filed: Jan. 12, 2004

(30) Foreign Application Priority Data
Jan. 11, 2003 (DE) 103 00 735.0

(51) Int. Cl. A61N 1/39; A61B 5/02; A61B 5/0402

(52) U.S. Cl. 607/7; 600/518; 600/500; 600/504

(57) ABSTRACT

The method and the device serve for detecting an anomaly in the cardiac activity of a patient and for carrying out a defibrillation. At least one sensor acquires at least one parameter that characterizes the cardiac activity of a patient. An automatic evaluation with respect to at least one parameter that characterizes the anomaly in the cardiac activity is carried out, and an alarm signal is generated if a limiting value for at least one parameter that characterizes the anomaly is exceeded. The defibrillator is realized in the form of a mobile unit and contains a voltage generator, a control unit and at least two electrodes.

The invention pertains to a method and a device for detecting an anomaly in the cardiac activity of a patient.

The increasing proportion of older persons in our society leads to a significantly higher number of cardiopulmonary diseases with sometimes life-threatening consequences. The most common disease of this type is myocardial infarction. This disease damages the myocardium and can ultimately lead to heart failure (CHF: Congestive Heart Failure). The changes to the cardiovascular system resulting thereof frequently also affect pulmonary parameters such that most of these patients are also dependent on artificial respiration. These patients are categorized as risk patients because the damaged heart has a tendency to ventricular fibrillation that ultimately leads to sudden cardiac death.

In the meantime, therapy methods have been developed for all sub areas of the diseases in question. Respiratory disorders can be effectively treated with CPAP devices or methods derived thereof, and ventricular fibrillation (VP) can be treated with defibrillation. Medicinal therapies are generally becoming less and less popular because extensive studies verified that these therapies are only successful in the short term, with proarrythmic effects predominating in the long term.

Due to its comparatively low cost and simple handling, the CPAP therapy is also suitable for home use and, in particular, paid for by health insurance companies. However, defibrillation is only slowly gaining acceptance. Implantable defibrillators (so-called ICDs) cost approximately [symbol] 20,000 and consequently are only authorized by health insurance companies after a person survives a VF attack. External defibrillators are becoming more and more popular in ambulatory treatments. However, external aid usually arrives too late due to the long driving times of emergency vehicles. Only about 2% of all VF patients in Germany currently survive their first attack. This indicates an urgent need for the development of new, less expensive and simpler methods that ensure a higher survival rate in this extensive group of patients.

A few years ago, so-called semiautomatic defibrillators that are also referred to as AEDs (Automatic External Defibrillators) or PADs (Public Access Defibrillators) were introduced on the market. Due to their automatic diagnosis function, these defibrillators can also be operated by medical laymen. The driving time for an emergency physician would no longer be a decisive factor and the probability of survival could be increased if these devices would be distributed as broadly as fire extinguishers. An extensive study at the Chicago O'Hare airport, for example, showed that the rate of successful reanimations can be increased to nearly 90% if the airport building is equipped with such devices and the personnel is instructed in their handling. In the year 2000 alone, 12 patients were successfully reanimated in this fashion at the above-mentioned airport. Risk patients, in particular, are able to purchase and install devices of this type in their homes such that they are constantly accessible to family members. Alternatively, the devices may be carried along by these risk patients.

The firms Medtronic and Lifecor recently introduced a different concept in the form of a wearable vest that contains a defibrillator. The defibrillator analyzes the EKG of the patient and, in case of an emergency, activates the electrodes that are also sewn into the vest in a fully automated fashion, pneumatically presses electrode gel on the electrode/skin boundary surface and defibrillates. A recently published FDA study shows that the survival rate increases to 25%. This result indicates that wearable external defibrillators represent a sensible alternative to existing concepts.

However, it should be noted that some of the patients do not survive an attack if the device is not worn at the time in question. A device of this type is unacceptable, in particular, during nighttime hours because the external components do not allow the patient to sleep restfully.

In addition, the technically complicated construction of these devices results in a price of approximately [symbol] 10,000 and consequently does not provide any significant economical advantages. The sole advantage can be seen in the fact that external devices represent an investment that can be transferred from one patient to another patient while implants are consumables that become the property of one patient. A transfer, for example, in the sense of a Bridging Device between transplant candidates consequently is legally questionable.

According to the state of the art, a therapy in the form of external defibrillation is applied in all circumstances, in which the patient is monitored. However, this state of the art does not provide sufficient protection, in particular, during nighttime hours and for solitary persons or persons who are not monitored with clinical equipment (clinics, nursing homes, etc.).

The present invention is based on the objective of improving a method of the initially described type in such a way that an activation of the device is achieved with a short time delay.

According to the invention, this objective is attained due to the fact that at least one parameter that characterizes the cardiac activity of a patient is determined by means of at least one sensor, the fact that at least one parameter that characterizes the anomaly in the cardiac activity of the patient is automatically evaluated, and the fact that an alarm signal is generated if a limiting value for at least one parameter that characterizes the anomaly in the cardiac activity is exceeded.

In the context of the invention, the term “exceeding a limiting value” is a general term and, for example, also includes the triggering of an alarm based on a fuzzy logic. For example, it is possible to generate an alarm signal if parameters are determined which are not individually evaluated as being critical, but altogether characterize a critical state that causes an alarm to be triggered.

The anomaly in the cardiac activity of a patient may be any deviation from the normal cardiac activity, for example, ventricular fibrillation, ventricular flutter, ventricular tachycardia and asystolia. However, any other anomalies in the cardiac activity may also be considered.

The alarm signal may consist, for example, of a signal that activates a defibrillator and can be perceived acoustically or optically, for example, simultaneously or in a delayed fashion. In addition, the term “alarm signal” also includes a signal that does not lead to an immediate activation of the defibrillator, but rather provides merely status information as to the fact that a limiting value has been exceeded. A signal of this type can be generated arbitrarily, for example, optically or acoustically.

According to one preferred embodiment of the invention, the signal measurement on the patient and the generation of an alarm signal are carried out in a spatially separated fashion. In this case, the signal measurement may be realized with mobile, portable sensors. The generation of an alarm signal may take place in the immediate vicinity of the patient, for example, on the nightstand, in a physician's room or nurses' station, an emergency dispatch center, at relatives or friends, etc.

The signal evaluation may take place on the patient or in a spatially separated fashion. The data transmission between the sensor(s) and the signal evaluation unit and between the signal evaluation unit and the means for generating an alarm signal may be realized arbitrarily, for example, in a wire-bound or wireless fashion.

Other advantageous embodiments of the method according to the invention form the objects of the dependent claims.

The anomaly in the cardiac activity of a patient may be, for example, a state of fibrillation, and the parameter that characterizes the anomaly in the cardiac activity may be a fibrillation parameter.

A practical measurement of the respective cardiac activity may be realized in the form of a metrological acquisition of an EKG signal. The acquisition of a pulse signal or a hemodynamics signal may be carried out alternatively or additionally.

A high degree of convenience can be achieved by acquiring the measuring values in the region of at least one adhesive pad. It would also be conceivable to acquire the measuring values in the region of at least one arm- or wristband, neckband, abdominal band or hip band.

A signal measurement in the immediate vicinity of the heart can be achieved by acquiring the measuring values in the region of a thoracic band.

The convenience can be additionally improved by caring out the sensory acquisition of measuring values and the evaluation of the measuring signals in a spatially separated fashion.

According to the invention, the measuring data acquired by the sensor are transmitted, in particular, in a wireless fashion to a signal evaluation unit. In this case, the patient essentially carries along only the sensors, as well as the data transmission means and a power supply. The signal evaluation unit and the means for generating alarm signals may be arranged at a remote location and realized, for example, in a stationary or portable fashion.

It would also be conceivable to carry out the sensory acquisition of measuring data and the evaluation of the measuring signals in a spatially adjacent fashion, wherein the results are transmitted to the signal evaluation unit in this case.

A compact design can be achieved by carrying out the acquisition of measuring data in the region of a respiratory mask.

The generated signal typically consists of an acoustic alarm signal. In addition, it is possible to generate an optical alarm signal.

It is particularly advantageous to store the values of at least one parameter that characterizes the cardiac activity of the patient. These values may be stored at an arbitrary location. For example, it is possible to store the data in a module that is carried along by the patient. It is also possible to store the data in a stationary memory, for example, in a server. The retrievable storage of this data provides the advantage that the course of the disease and the state of the patient before the onset of an emergency can be studied in order to simplify the decisions regarding additional measures to be taken. The values may be retrievably stored in a memory. It would also be conceivable for the memories to transmit the values actively.

The values of the at least one parameter that characterizes the cardiac activity of a patient may be directly transmitted from the sensor to the memory. It would also be conceivable to initially transmit the values from the sensor to a signal evaluation unit and then from the signal evaluation unit to the memory. For example, the transmission from the signal evaluation unit to the memory may take place in a wireless fashion.

According to one preferred embodiment of the invention, a flag signal that causes an alarm signal to be delivered is generated once a limiting value is exceeded. The flag signal may be generated, for example, by a signal generator and cause a signal transmitter to deliver the alarm signal.

The flag signal may be transmitted in a wire-bound or wireless fashion. The transmission technique is not subject to any limitations. The flag signal may be transmitted via short-range data transmission, in particular, Bluetooth, or via long-range data transmission, in particular, by means of a telephone, a mobile radiotelephone or a radio link.

When using short-range data transmission, the alarm signal can be delivered, for example, optically and/or acoustically by a signal transmitter that is situated within the reception range of the flag signal. Naturally, it would also be possible to deliver the flag signal to the signal transmitter in a wire-bound fashion.

When using long-range data transmission, it is possible to transmit the flag signal to a mobile terminal that delivers the alarm signal once the flag signal is received, for example, to a radio apparatus, a cellular phone or a PDA. This makes it possible to deliver an alarm signal to any arbitrary location, in particular, independently of the current location of the patient. The alarm signal may be transmitted, for example, to family members, an emergency physician or an emergency dispatch center. It would be possible, for example, to place a local signaling device that delivers the alarm signal in the residence of the patient or of family members.

It is possible, for example, to transmit the alarm signal in the form of a telephone voice message or in the form of a SMS.

According to the invention, it is also possible to transmit the stored values of the at least one parameter that characterizes the cardiac activity of a patient or information on a storage location, from which the values can be retrieved, together with the flag signal. The transmission of this information may take place simultaneously with or a certain time after the transmission of the flag signal or even in dependence thereon. This embodiment of the invention not only makes it possible to deliver an alarm signal to the respective receiver, but also information on the state of the patient over an extended period of time, in particular, before the onset of the emergency. This assists the treating physician in deciding on further treatment steps.

For example, it would be conceivable to transmit the values of the parameter that characterizes the cardiac activity of a patient to the physician who receives the alarm signal. These values can be immediately displayed, for example, on a PDA. It would also be possible to display, for example, the EKG on the PDA.

These values can be transmitted before, during or after the transmission of the flag signal. This applies correspondingly to the information on the storage location which, for example, may be implicitly contained in the alarm signal. It would be possible, for example, that the alarm signal consists of the name of the patient or a device number of a defibrillator, and that the stored data can also be found under these designations. The data may be stored, for example, on a server.

In another embodiment of the invention, this applies analogously to patient data or information on a storage location, from which patient data can be retrieved. In this case, the addressee of the alarm signal is able to access important patient data in order to ensure an optimal treatment therapy. The patient data may comprise, for example, data on prior diseases, medications taken, allergies to medications, etc.

The values of the signal that characterizes the cardiac activity of a patient and/or the patient data can, for example, be downloaded or inspected with the aid of an Internet browser.

In another embodiment of the invention, the receiver of the alarm signal is able to initiate a wireless data transmission in order to activate a defibrillator. It would be conceivable to utilize a mobile radiotelephone for this purpose.

In another embodiment of the invention, it is determined if and how the patient is moving, and this information is used for determining whether a limiting value is exceeded or not together with the parameters that characterize the cardiac activity of a patient. A particularly accurate determination of the exceeding of a limiting value can be achieved in this fashion because the movements of the patient are taken into account in calculating the limiting value.

Another objective of the present invention consists of constructing a device of the initially described type in such a way that an improved utilizability is achieved.

According to the invention, this objective is attained with a device for detecting an anomaly in the cardiac activity of a patient which comprises at least one sensor for acquiring at least one signal that characterizes the cardiac activity of a patient, at least one signal evaluation unit to which the sensor is connected and a signal transmitter to which the signal evaluation unit is connected, wherein the signal evaluation unit is provided with an analyzer for detecting if at least one parameter that characterizes the anomaly in the cardiac activity is exceeded.

The anomaly in the cardiac activity of a patient may be any heart disorder, for example, dysrhythmia or another abnormal condition. For example, the anomaly in the cardiac activity of a patient is a state of fibrillation, and the parameter that characterizes the anomaly is a fibrillation parameter.

According to one preferred embodiment of the invention, the sensor(s) is/are realized in the form of (a) portable, mobile sensor(s). The signal evaluation unit may also be arranged on the patient or at a different location and, for example, be realized stationarily. According to the invention, it is preferred to spatially separate the signal transmitter from the patient, i.e., the signal transmitter is not arranged directly on the patient. This means that the sensor(s) and, if applicable, the signal evaluation unit can be carried along by the patient. According to preferred embodiments of the invention, this does not apply, however, to the signal transmitter. The signal transmitter may still be situated in the vicinity of the patient, for example, on the bed or in the vicinity of the bed, or at a remote location, for example, in a physician's room, an emergency dispatch center or in the residence of friends or relatives.

Advantageous embodiments of the device form the objects of the dependent claims.

According to another embodiment of the invention, the signal transmitter can be activated by a signal generator.

At least one sensor for acquiring at least one signal that characterizes the cardiac activity of a patient may be connected to the control unit, wherein the sensor is connected to an evaluation unit for determining if a limiting value for at least one fibrillation parameter is exceeded, and wherein the evaluation unit is connected to a signal transmitter that can be activated by a signal generator.

According to another embodiment of the invention, the device according to the invention for detecting an anomaly in the cardiac activity of a patient consists of a defibrillator that is realized in the form of a mobile unit and provided with a voltage generator, a control unit and at least two electrodes, wherein at least one sensor for acquiring at least one signal that characterizes the cardiac activity of a patient is connected to a signal evaluation unit, wherein the signal evaluation unit is provided with an analyzer for determining if a limiting value for at least one fibrillation parameter is exceeded, and wherein the signal evaluation unit is connected to a signal transmitter that can be activated by a signal generator.

The utilization and the evaluation of a sensor for acquiring at least one parameter that characterizes the cardiac activity of a patient, as well as the evaluation of the measuring values with respect to the exceeding of a limiting value for the at least one parameter, for example, a fibrillation parameter, make it possible to detect the time for a successful device activation in a timely fashion with respect to metrological considerations, and to generate the required control signals by means of the assigned signal generator and the signal transmitter. An activation of the defibrillator may be realized directly with the signals generated by the signal transmitter. It is also possible that the signal transmitter directly transmits only a status signal that is used for activating the defibrillator by means of a series-connected signal evaluation unit.

It is preferred that the sensors continuously monitor the signals that characterize a cardiac activity of a patient. In one preferred embodiment of the invention, the evaluation unit comprises digital signal processors.

A compact device can also be achieved by realizing the signal evaluation unit as part of the control unit.

A positioning flexibility can be achieved by arranging the signal evaluation unit and the control unit in a spatially separated fashion.

The sensor of the device according to the invention may be designed for acquiring at least one EKG signal, pulse signal and/or hemodynamics signal.

The sensor may be arranged in the region of at least one adhesive pad, wristband, neckband, thoracic band, abdominal band or hip band, as well as in the region of a respiratory mask. For example, it would be conceivable to provide several adhesive pads and/or several wristbands with corresponding sensors.

The sensor may be arranged adjacent to or spatially separate from the signal evaluation unit. In a further embodiment of the invention, the sensor is coupled to the signal evaluation unit in a wireless fashion. In this case, the patient only carries along the sensor(s) and the sensor signals are transmitted to the signal evaluation unit. It would also be possible for the patient to carry along the sensors and the signal evaluation unit, wherein the result of the signal evaluation is then transmitted to a signal generator, for example, in a wireless fashion. This provides the advantage that a data transmission only needs to take place if the signal evaluation indicates that a limiting value is exceeded.

Consequently, it is possible to realize an embodiment, in which the patient merely carries along one or more mobile, portable sensors, a data transmission unit and a power supply while the other components of the device are arranged in a spatially separated fashion. The alarm signal may be generated at a suitable location, for example, in a physician's room, an emergency dispatch center, at the residence of relatives or in the vicinity of the patient, for example, on the nightstand.

The signal transmitter may be realized arbitrarily. It would be conceivable to utilize, for example, optical or acoustical signal transmitters.

In another preferred embodiment of the invention, a memory is provided for storing the values of the at least one parameter that characterizes the cardiac activity of a patient and/or patient data. The memory may consist of a mobile memory, i.e., it can be carried along by the patient. However, the memory may also be arranged stationarily and, for example, be realized in the form of a server. The sensor(s) may continuously transmit the acquired data to the memory. The data may be transmitted in a wire-bound or wireless fashion. It is advantageous to record the data for a predetermined duration (e.g., over several weeks or months) such that the medical personnel are able to evaluate the course of the disease and the cause for the current condition.

It is also possible to provide means for transmitting a flag signal delivered by the signal generator in a wire-bound or wireless fashion. Arbitrary transmission techniques may be considered, for example, Bluetooth or mobile radiotelephony.

It would also be conceivable to transmit the values of the parameter that characterizes the cardiac activity of the patient in real-time. This may be advantageous with respect to a functional check. These values may be transmitted by the sensor(s) or an evaluation unit connected thereto.

It would also be conceivable to provide motion sensors that make it possible to ascertain whether or not the patient is moving. The signals acquired by these sensors can be used for determining whether a limiting value is exceeded or not together with the acquired parameters.

The sensors for acquiring the parameter that characterizes the cardiac activity of the patient may consist of defibrillation electrodes. It would also be conceivable to provide sensors for acquiring the parameters that characterize the cardiac activity in addition to the defibrillation electrodes.

It would be possible to provide means for obtaining information on the current location of the patient in order to enable the respondent to render aid as quickly as possible. This information may form part of, for example, the flag signal generated by the signal generator and be transmitted to the receiver of the alarm signal by the signal transmitter.

The device according to the invention may permanently or within certain time intervals receive information on the current location of the device and consequently the patient, wherein this information is stored and transmitted on demand. Such a reception and storage unit may be integrated, for example, into the signal evaluation unit. Information of this type may consist, for example, of positional information such as the coordinates or identifications of radio cells or Location Areas of a mobile radiotelephone network which are made available by a network transmitter. In this case, the device according to the invention is equipped with means that make it possible to acquire positional data. Other localization techniques known from the field of navigation systems may also be used. The precise determination of the current location may be realized, for example, with the aid of GPS. The device according to the invention can be equipped with a corresponding receiver for this purpose. The positional data can be stored and, if so required, transmitted to an addressee or retrieved.

Another embodiment of the invention comprises means that carry out a self-test of the device continuously or within certain time intervals.

The sensors, the electrodes and/or the evaluation unit can preferably be integrated into garments (brassieres, slips, belts, etc.). The sensors and/or the electrodes, in particular, may be realized such that they do not have to be removed when the garments are laundered. The evaluation unit and any other components of the device are preferably realized such that they can be removed from the garment.

The invention also proposes to realize the power supply for the components of the device, in particular, the components carried along by the patient, in the form of a rechargeable battery.

Embodiment of the invention are schematically illustrated in the figures. The figures show:

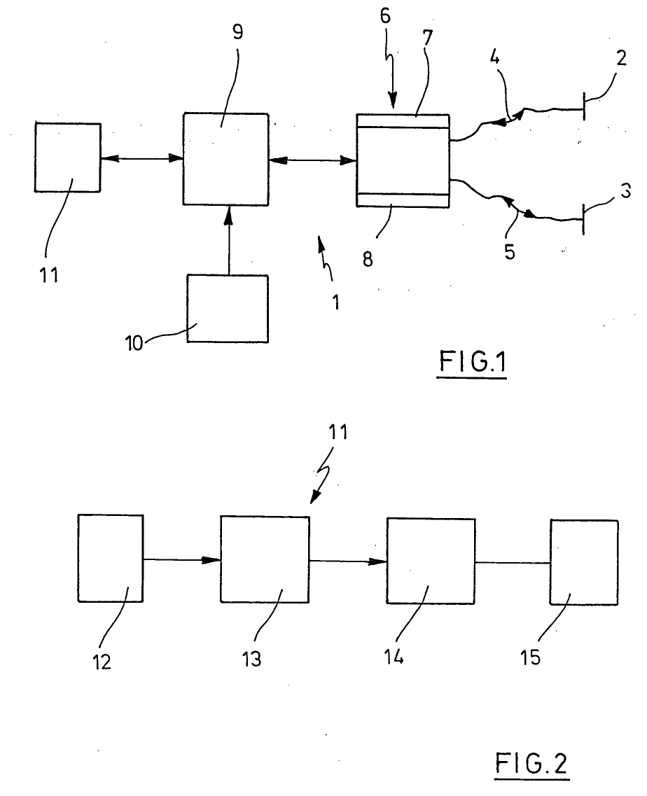

FIG. 1 a schematic block diagram for elucidating the basic design of a defibrillator with a monitoring device;

FIG. 2 a block diagram for elucidating the basic design of the monitoring device;

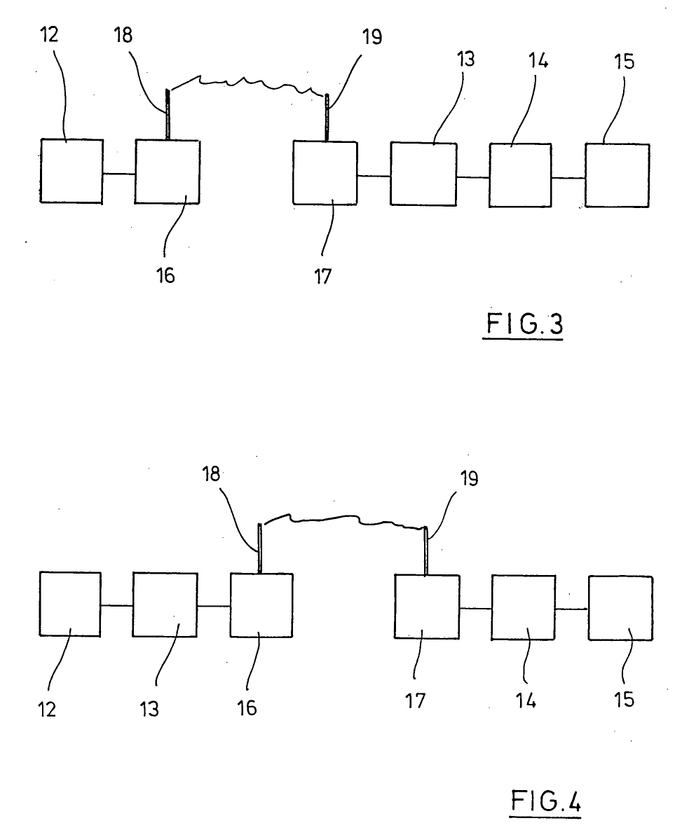

FIG. 3 an embodiment that is modified in comparison with FIG. 2, wherein a sensor and a signal evaluation unit are arranged spatially separate from one another and connected via a wireless link, and

FIG. 4 an additionally modified embodiment, in which the signal evaluation unit is arranged spatially adjacent to the sensor and a wireless transmission of already pre-processed data takes place.

FIG. 1 shows the basic design of a defibrillator 1. The defibrillator 1 contains two electrodes 2, 3 that are connected to a base unit 6 via connecting lines 4, 5. The base unit 6 is provided with a shock transmitter 7 and a detection unit 8.

The shock transmitter 7 is designed for applying electric shocks to the electrodes 2, 3. The detection unit 6 serves, in particular, for recording electric signals in the region of the electrodes 2, 3, for example, an electrocardiogram. The detection unit 8 assists, in particular, in delivering the electric shock at the optimal time.

The base unit 6 is connected to a control unit 9 that is able to evaluate the signals of the detection unit 8. It is also possible for the detection unit 8 to act upon the shock transmitter 7 directly. The control unit can be manually actuated with an operating unit 10.

A monitoring device 11 that determines at least one parameter that characterizes the cardiac activity of the patient is also connected to the control unit 9. It would also be conceivable, in particular, to realize the monitoring device 11 in the form of a mobile EKG unit. The monitoring device 11 may be positioned, for example, in the region of an adhesive pad, in the region of a wristband or in the region of a thoracic band.

FIG. 2 shows a more detailed representation of the design of the monitoring device 11. The monitoring device 11 essentially consists of a sensor 12, a signal evaluation unit 13, a signal generator 14 and a signal transmitter 15. The signal evaluation unit 13 carries out a comparison as to the fact whether or not the measured parameters characterize a state of fibrillation.

If such a state is detected, a suitable flag signal is generated by the signal generator 14 and delivered by the signal transmitter 15. The signal transmitter 15 may be either directly connected to the control unit 9 or the signal delivered by the signal transmitter 15 is indirectly utilized for activating the defibrillator 1.

In the embodiment according to FIG. 3, the sensor 12 is arranged spatially separate from the signal evaluation unit 13 and the downstream components. The sensor 12 is merely coupled to a transmitter 16, wherein an antenna 18 serves for producing a wireless link between the transmitter and a receiver 17 that is provided with an antenna 19. Such an embodiment allows a simple realization of the acquisition unit that contains the sensor 12 and can be comfortably worn by the patient.

According to one special embodiment, the signal transmitter 15 is realized, in particular, in the form of an acoustical signal transmitter or an optical signal transmitter. The thusly generated alarm signal can be detected and used for activating the defibrillator 1.

According to another embodiment, it is possible to arrange the detection unit 8 at least partially in the region of a respiratory mask used for CPAP therapy. This simplifies the attachment of the monitoring device 11 for the patient because another separate part is no longer required.

In the embodiment according to FIG. 4, the signal evaluation unit 13 is arranged spatially adjacent to the sensor 12. This embodiment makes it possible to reduce the volume of data to be transmitted between the transmitter 16 and the receiver 17. This also results in a reduced power consumption of the transmitter 16 and, if applicable, in an extended battery life.

1. A method for detecting an anomaly in the cardiac activity of a patient wherein at least one sensor (12) determines at least one parameter that characterizes the cardiac activity of a patient, an automatic evaluation with respect with respect to at least one parameter that characterizes the anomaly in the cardiac activity is carried out, and in that an alarm signal is generated if a limiting value for at least one parameter that characterizes the anomaly in the cardiac activity is exceeded.

2. The method according to claim 1, wherein the anomaly in the cardiac activity of a patient is a state of fibrillation and the parameter that characterizes the anomaly in the cardiac activity is a fibrillation parameter.

3. The method according to claim 1, wherein a metrological acquisition of an EKG signal, a pulse signal and/or a hemodynamics signal is carried out.

4. The method according to claim 1 wherein the acquisition of measuring values is carried out in the region of at least one adhesive pad, wristband, neckband, thoracic band, abdominal band, hip band and/or in the region of a respiratory mask.

5. The method according to claim 1 wherein the sensory acquisition of measuring data and the evaluation of the measuring signals are spatially separated.

6. The method according claim 1, wherein the sensory acquisition of measuring data and the evaluation of the measuring signals are carried out spatially adjacent to one another, and the results of the signal evaluation are transmitted to a different location.

7. The method according to claim 1 wherein the measuring data acquired by the sensor (12) are transmitted in a wireless fashion to a signal evaluation unit (13), or the results of the signal evaluation (13) are transmitted in a wireless fashion to a signal generator (14).

8. The method according to claim 1 wherein an acoustical and/or optical alarm is generated.

9. The method according to claim 1 wherein the alarm signal comprises a control signal that causes a direct activation of a defibrillator.

10. The method according to claim 1 wherein the values of the at least one parameter that characterizes the cardiac activity of a patient are stored.

11. The method according to claim 1 wherein a flag signal that causes the delivery of the alarm signal is generated if a limiting value is exceeded.

12. The method according to claim 11, wherein the flag signal is transmitted in a wire-bound or wireless fashion.

13. The method according to claim 12, wherein the flag signal is transmitted by means of short-range data transmission, in particular, Bluetooth, or by means of long-range data transmission, in particular, a telephone or mobile radiotelephone.

14. The method according to claim 11 wherein the stored values of the at least one parameter that characterizes the cardiac activity of a patient or information on a storage location, from which the values can be retrieved, are transmitted together with the flag signal.

15. The method according claim 11 wherein patient data or information on a storage location, from which the patient data can be retrieved, are transmitted together with the flag signal.

16. The method according claim 1 one wherein it is determined if and how the patient is moving and this information is used for determining if a limiting value is exceeded together with the parameters that characterize the cardiac activity of a patient.

17. A device for detecting an anomaly in the cardiac activity of a patient, comprising at least one sensor (12) for acquiring at least one signal that characterizes a cardiac activity of a patient, at least one signal evaluation unit (13) to which the sensor (12) is connected and a signal transmitter (15) to which the signal evaluation unit (13) is connected, wherein the signal evaluation unit (13) is provided with an analyzer for determining if a limiting value for at least one parameter that characterizes the anomaly in the cardiac activity is exceeded.

18. The device according claim 17, wherein the anomaly in the cardiac activity of a patient is a state of fibrillation, and in that the parameter that characterizes the anomaly in the cardiac activity is a fibrillation parameter.

19. The device according claim 17 wherein the signal transmitter (15) can be activated by a signal generator (14).

20. The device according claim 17 wherein the device is realized in the form of a mobile unit and used for defibrillation purposes, and the device contains a voltage generator, a control unit (9) and at least two electrodes (2, 3).

21. The device according claim 20, wherein the signal evaluation unit (13) forms part of the control unit (9).

22. The device according to claim 20, wherein the signal evaluation unit (13) is spatially separated from the control unit (9).

23. The device according to claim 17 the sensor (12) is arranged adjacent to or spatially separate from the signal evaluation unit (13).

24. The device according claim 17 wherein the sensor (12) and the signal evaluation unit (13) are connected via a wireless link.

25. The device according claim 17 wherein a memory is provided for storing the values of the at least one parameter that characterizes the cardiac activity of a patient and/or patient data.

26. The device according claim 17 wherein the signal transmitter (15) and the signal generator (14) are connected in a wire-bound or wireless fashion.

27. The device according claim 17 wherein the motion sensors are provided for determining if and how the patient is moving.

28. The device according claim 17 wherein the sensor (12) for acquiring at least one signal that characterizes a cardiac activity of a patient of comprises defibrillator electrodes.

29. The device according claim 17 means are provided for obtaining information on the current location of the patient.
